(19) United States
(12) Patent Application Publication
(10) Pub. No.: US 2008/0262574 A1
(43) Pub. Date: Oct. 23, 2008

(54) Use of low intensity light therapy for the treatment of various medical conditions

(75) Inventors: Nancy M. Briefs, Nashua, NH (US); Michail M. Pankratov, Waltham, MA (US)

Correspondence Address: LAHIVE & COCKFIELD, LLP;FLOOR 30, SUITE 3000, ONE POST OFFICE SQUARE, BOSTON, MA, 02109, US

(73) Assignee: Eleme Medical Inc., Merrimack, NH (US)

(21) Appl. No.: 12/082,699

(22) Filed: Apr. 11, 2008

Related U.S. Application Data
(60) Provisional application No. 60/922,865, filed on Apr. 11, 2007.

(51) Int. Cl. A61B 18/18; A61H 7/00

(52) U.S. Cl. 607/88; 601/6

(57) ABSTRACT

The invention relates to the use of low intensity light therapy for the treatment of various medical conditions, either alone, or in combination with a second procedure.

### RELATED APPLICATION

This application claims priority to, and the benefit of, co-pending U.S. Provisional Application U.S. Ser. No. 60/922,865, filed Apr. 11, 2007, for all subject matter common to both applications. The disclosure of said provisional application is hereby incorporated by reference in its entirety.

### FIELD OF THE INVENTION

The present invention relates to the use of low intensity light therapy for the treatment of various medical conditions, either alone, or in combination with a second procedure.

### BACKGROUND OF THE INVENTION

Therapeutic effects of Low Level Light Therapy (LLLT) or Low Intensity Light Therapy (LILT) are generally well known. For example, the ability of monochromatic light to modify cellular function of living tissue, enhance healing and restore its normal function is a basis for the treatment of dermatological, musculoskeletal, soft tissue and neurological conditions. The therapy is being promoted for the treatment of intractable pain, reduction of inflammation and edema, assistance in wound healing and treatment of sports injuries. These cellular and biochemical changes produce beneficial therapeutic results in chronic wound healing, improvements in acute athletic injuries and chronic conditions, including carpal tunnel syndrome, TMJ, arthritis, and neuropathies (for review see Vladimirov et al. (2004) Biochemistry (Moscow) 69:81). LILT has demonstrated efficacy in improving wound healing, reducing edema, and relieving pain of various origins.

Cellulite is not only prevalent in the overweight. It is estimated that between 90% and 98% of the total female adult population have had, will have, or currently do have, a desire to reduce an amount of cellulite. The male population also struggles with its desire to reduce cellulite amounts.

Once cellulite begins, it is chronic and spontaneously irreversible (without specific treatment). If extracted and chemically analyzed, cellulite is a jelly-like substance composed of water and fat. Cellulite becomes trapped in tissues. Unlike ordinary fat, cellulite is not readily available to the organism. Because it is trapped, it is relatively isolated from the natural processes of absorption and elimination.

Secondarily, cellulite interferes with circulation. The formation of cellulite partially occurs due to venous and lymphatic stagnation. The vicious cycle is then closed and tends to perpetuate itself.

Cellulite is a generalized condition that is fundamentally based on nutrition of the cells. As fat cells increase in volume and infiltration, they add to an already existing circulatory disturbance. This leads to a decrease in the local metabolic rate, which helps perpetuate the cellulite cycle. The sticky, fatty masses find locations in the connective tissue (otherwise known as interstitial tissue) between the skin and muscle in the subcutaneous layer. When there is a disturbance in the connective tissue, additional problems result. The additional problems occur in part because the connective tissue envelops all internal organs including the lymphatic system, which contributes to the pathway of all nutrients and wastes to and from the cells.

Increases of cellulite deposits literally waterlog the connective tissue, which eventually breaks down. The connective tissue then attempts to repair itself, eventually forming a fibrous scar tissue. The newly thickened tissue then traps more fat and water, which again develops cellulite.

A number of non-invasive methods currently exist that attempt to reduce cellulite on a temporary basis. These methods primarily involve the application of topical agents to reduce the appearance of cellulite on the thigh, which have been reported to be effective as a temporary means of cellulite reduction. Other non-surgical but invasive means of cellulite reduction currently being investigated include methods of fat reduction by direct injection of antibodies to native fat, or injection of agents that otherwise accelerate resorption of fat. Another alternative for temporary improvement in the appearance of cellulite is a mechanical massager with controlled suction (e.g., Endermologie® (LPG Systems)). Other systems seek to increase superficial bloodflow to reduce the appearance of cellulite by using a low intensity laser (810 nm) with topical cooling and mechanical massaging (e.g., TriActive™ (Cynosure)).

A number of invasive methods currently exist that also attempt to reduce cellulite. These methods include targeted surgical adipose tissue resection in the fatty layers of the subcutaneous tissue. This method is considered an invasive procedure with potential risks and complications including pain, numbness, surface contour irregularity, and death.

More recently, methods involving use of light therapy have also been developed for reducing cellulite, however, most of the methods currently available, result in excessive elevating the temperature of treated tissue to the level of a high fever, which is undesirable. For example, the VelaSmooth™(Syneron) system combines bi-polar radio frequency and broadband infrared light (ELOS™ technology) with mechanical massaging and suction. Broad band infrared light provides bulk tissue heating and conducted RF energy is designed to penetrate up to 15-mm deep into the tissue causing heating of subcutaneous fat.

### SUMMARY OF THE INVENTION

The present invention provides a method of treating various medical conditions using low intensity light therapy (LILT), which avoids undesirable effects such as, for example, excessive elevation of tissue temperature. The method of the invention provides a means to remove discrete subcutaneous adipose tissue and improve the appearance of cellulite.

Exemplary medical conditions which may be treated using methods of the invention include, but are not limited to, for example, cellulite, poor blood circulation, inflammation, pain, tissue damage wounds.

In one embodiment, a method of the present invention is used for reducing cellulite and superficial fat in a subject in need thereof. The method includes treating a subject with a first wavelength of low intensity light for a duration of time that is sufficient for increasing the permeability of cell membranes of adipocytes and at least a second wavelength or plurality of distinct wavelengths of low intensity light for a duration of time that is sufficient for causing liquefaction of fat in the adipocytes, thereby resulting in cellulite reduction. In one embodiment, the subject is exposed to the first wavelength, a second wavelength, and any subsequent wavelengths sequentially, e.g., the first wavelength is used first followed by the second wavelength, followed by a third, etc., or alternating, e.g., the second wavelength is used first, followed by the first wavelength, switching back and forth in an alternating manner. In another embodiment, the first wavelength, the second wavelength, and any additional wavelengths are used simultaneously to achieve the desirable result, e.g., cellulite and fat reduction.

In another embodiment, a method of the present invention for reducing cellulite includes treating a targeted area in a subject in need thereof with a first wavelength of low intensity light for a duration of time that is sufficient for increasing the permeability of cell membranes of adipocytes and at least a second wavelength of low intensity light for a duration of time that is sufficient for causing liquefaction of fat in the adipocytes, in combination with a method resulting in a moderate increase in tissue temperature of the targeted area, wherein the combination results in cellulite reduction in the targeted area.

In another embodiment, the method of the invention comprises increasing the permeability of a cell membrane of an adipocyte of the targeted area; increasing the temperature of the adipocyte such that lipid liquefaction occurs; and evacuating liquefied fat from the adipocyte. Such a method may be used to reduce or prevent cellulite or superficial fat from a targeted area of a subject. In one embodiment, the targeted art of the subject is treated with a wavelength of low intensity light, including but not limited to 600 to about 699 nm or 900 to 950 nm. In one embodiment, the wavelength of low intensity light is about 650 nm or about 915 nm. In one embodiment, the evacuation of the liquefied fat is achieved using a massage system, such as, but not limited to, a vacuum massage system.

In yet another embodiment, the invention provides a method of achieving one or more therapeutic effects selected from improving skin elasticity, reducing a circumference, and increasing collagen production in a treated area of a subject, said method comprising increasing the permeability of a cell membrane of an adipocyte of the targeted area; increasing the temperature of the adipocyte such that lipid liquefaction occurs; and evacuating liquefied fat from the adipocyte, In one embodiment, the targeted art of the subject is treated with a wavelength of low intensity light, including but not limited to 600 to about 699 nm or 900 to 950 nm. In one embodiment, the wavelength of low intensity light is about 650 nm or about 915 nm. In one embodiment, the evacuation of the liquefied fat is achieved using a massage system, such as but not limited to a vacuum massage system, on the treated area of the subject.

The methods of the invention may be used to reduce or prevent cellulite or superficial fat (or achieve a therapeutic effect) over the course of a time period following treatment. In one embodiment, reduction or prevention of cellulite or superficial fat improves over the course of at least 1 month. In one embodiment, reduction or prevention of cellulite or superficial fat improves over the course of at least 2 months. In one embodiment, reduction or prevention of cellulite or superficial fat improves over the course of at least 3 months.

In one embodiment, a moderate increase in tissue temperature is up to about 102 to about 106° F. It is contemplated that any suitable means of increasing tissue temperature may be used in the methods of the invention.

The low intensity light used in the methods of the invention in accordance with some embodiments may be continuous, and in accordance with other embodiments may be pulsed, so long as it achieves the desired result.

In one embodiment, the method of reducing cellulite further comprises use of a massage system, wherein the therapeutic effect resulting from the combination of the low intensity light therapy and the massage system is a synergistic effect. The massage system used in the methods of the present invention may include either a vacuum device, a mechanical device, or both.

In yet another embodiment, a method of the present invention is used for achieving one or more therapeutic effects selected from the group consisting of: improving local blood and lymphatic circulation; improving metabolic activity of cells; increasing collagen production; reducing inflammation; improving cell survival; and increasing tissue oxygenation. Accordingly, in a method according to the present invention, one or more of these therapeutic effects are achieved by treating a subject with a first wavelength of low intensity light and/or a second wavelength of low intensity light, for a duration of time sufficient to achieve one or more of the aforementioned therapeutic effects.

In one embodiment, the duration of time used in the methods of the invention is a predetermined duration of time required to achieve the desired result or treatment.

In one embodiment, the methods of the invention comprise the use of a radiation-emitting device. The radiation-emitting device can emit radiation in the visible (e.g., light) and/or infrared wavelengths. In one example embodiment, the radiation-emitting device emits radiation in a concentric combination of infrared and visible laser light. Alternatively, the wavelengths can be emitted by LEDs or equivalent light emitting devices in the visible and/or infrared light ranges.

In one embodiment, the vacuum massage comprises use of an automated mechanical massaging mechanism. The step of massaging the exposed skin can be executed by massaging from points distal from a heart of the patient to points proximal to the heart of the patient to encourage fluid flow toward the heart. Further, the step of massaging the exposed skin can include massaging in predetermined patterns of motion.

In one embodiment, the first and the second low intensity light wavelengths used in the methods of the invention are predetermined wavelengths.

In a particular embodiment, the first wavelength of low intensity light used in the methods of the invention is about 650 nm and the second wavelength of low intensity light used in the methods of the invention is about 900+/−20 nm, which includes about 915 nm.

However, the first and the second wavelengths used in the methods of the invention can be any low intensity light, which achieves the desired therapeutic effect, e.g., cellulite and superficial fat reduction or treatment of other medical conditions.

The invention further provides a classification system for determining improvements in cellulite reduction. Methods and compositions associated with the Cellulite Classification Tool (CCT) are also encompassed within the invention.

### DETAILED DESCRIPTION OF THE INVENTION

The present invention generally relates to methods of treating a selected patient condition, and preferably relates to tissue treatment, using low light intensity therapy, either alone or in combination with a second method. In a particular embodiment, a method according to the present invention reduces cellulite and superficial fat in a subject in need thereof. The present invention provides a non-destructive, modulating action of low intensity light therapy in combination with moderate temperature elevation in the irradiated tissue, as a basis of a dual-band light therapy for improving the appearance of cellulite. The dual band wavelength treatment is combined with a massage system for reducing cellulite in a subject.

Cellulite represents the excessive accumulation of fat cells within areas of connective tissue (in the subcutaneous layer) whereby additional microcirculatory and biochemical changes have taken place. Cellulite is a skin condition marked by enlarged adipocytes/lobules which are trapped in a fibrosed connective tissue network of fibrous septae. This localized form of fat tends to affect the thighs, the medial side of the knees, the buttocks, and the medial and outer sides of the arms, the nape, abdomen, flank, and the ankles. In cellulite states, the fundamental substance of the connective tissue increases its degree of fluid retention and viscosity therefore resulting in poor capillary exchange, which interferes with the mobilization of the excess deposits of fatty tissue. The present invention provides a method for stimulating restoration of healthy cell activity by addressing both the physical manifestations of cellulite and its underlying causes.

In studying the effects of LILT on adipose tissue, it was discovered that adipose cells exposed to low-level light at 635 nm wavelength developed a transitory pore in the adipocyte membrane (see, for example Solarte et al. (2002) SPIE Proceedings 4829:961 and Neira et al. (2006) Clin Plast Surg) 33:117). Light exposure and subsequent fat evacuation from the cell did not destroy the adipocytes but left them intact and in good condition. In addition, low level 900 nm wavelengths, including 900 or 915 nm, have been shown to have preferential absorption in lipids (Medrado et al. (2006) Lasers Med Sci 21:19; and Anderson et al. (2006) Lasers Med Surg 38:913). The 900+/−20 nm, which includes about 915 nm, wavelength penetrates well into the tissue with even less scattering than 650 nm but gets absorbed by the lipids in fat. The temperature inside the adipocytes gets slightly elevated causing fat liquefaction. The 650 nm and 915 nm wavelengths are also known to stimulate collagen regeneration and improve or restore blood and lymphatic circulation.

The present invention provides a method for increasing cell membrane permeability of an adipocyte in cellulite, liquefying lipids in the adipocyte, and evacuating the lipids from the adipocyte through the permeated cell membrane. The aforementioned combination results in a number of improved and unexpected effects, including, but not limited to, reduction in cellulite. By combining the low intensity light with massage, cellulite is unexpectedly reduced, and continues to be reduced for at least several months following treatment.

The invention is based on increasing the cell membrane permeability of an adipocyte, i.e., fat cell, to allow lipids to be released from the cell, thus reducing cellulite formation. Preferably, the adipocyte is found within cellulite in a target area of a subject. The method is achieved by increasing the permeability of a cell membrane of an adipocyte and increasing the temperature of the adipocyte such that lipid liquefaction occurs. Following lipid liquefaction, the lipids from the adipocyte are evacuated from the cell, thus decreasing the size of the cell and restoring healthy cell activity. Massaging the target area serves to move interstitial fluid toward the lymphatic system. By reducing cellulite in accordance with the method of the invention, a number of beneficial therapeutic effects may be achieved, including, but not limited to, improved skin elasticity, a reduction in the circumference of the target area (e.g., thigh), and an increase in collagen production.

In one embodiment, liquefaction of the lipids is achieved by treating a target area (having cellulite) with both a 915 nm wavelength and a 650 nm wavelength in combination with manual manipulation of the treated area, including, but not limited to, mechanical massage. In one embodiment, the permeability of the adipocyte is achieved by treating the targeted area of a subject with low intensity light, such as a wavelength ranging from 600 to 699 nm, including, but not limited to, 650 nm. The 650 nm wavelength was selected for its action in modifying permeability of the adipocytes membranes and enhancement of fat emulsification. This wavelength falls into an optical window where there is relatively minimal absorption in the skin components that allows for good penetration to the subcutaneous fat level. In one embodiment, a longer low intensity light wavelength, including, but not limited to, 900+/−20, which includes about 915 nm, penetrates the fat cells and is absorbed by the lipids, such that lipid liquefaction occurs. In one embodiment, the liquefied fat is evacuated from the adipocyte using a pressure means, such as massaging of the targeted area of the subject. Evacuation of liquefied fat from the inside of adipocytes into the intracellular space may also be facilitated by continuous vacuum massage.

It should be noted that the treatment of cellulite described herein may be achieved using other means that achieve adipocyte cell membrane permeability, lipid liquefaction within the adipocyte, and evacuation of the lipids from the adipocyte for reducing cellulite.

The term “targeted area” or “target area” as used herein, refers to a region, i.e., body tissue, of a subject having cellulite. Cellulite may be characterized as dimpled, orange-peel-like skin that is bumpy and lax. Such skin characteristics are outward symptoms of cellulite. The targeted area is subject to the methods of the invention for therapeutic improvement, including, but not limited to, reduction in cellulite, prevention in cellulite, reduction in superficial fat, and/or prevention of additional superficial fat. Examples of targeted regions which may be improved using the methods described herein include, but are not limited to, thighs, the medial side of the knees, the buttocks, and the medial and outer sides of the arms, the nape, abdomen, flank, and the ankles.

Exemplary medical conditions which may be treated using methods of the invention include, but are not limited to, for example, cellulite, poor blood circulation, inflammation, pain, tissue damage, wounds, and superficial fat reduction. Additional beneficial therapeutic effects which may result from the method of the invention include, but are not limited to, improved skin elasticity, a reduction in the circumference of the target area (e.g., thigh), and/or an increase in collagen production.

Accordingly, in one embodiment, a method of the present invention reduces cellulite and/or superficial fat by application of low intensity light to an area targeted for, e.g., cellulite and/or superficial fat reduction, at a predetermined wavelength, or combination of wavelengths, intervals, periods, and patterns. The application of infrared radiation can optionally follow. Subsequent to the application of infrared radiation, the tissue treatment system massages the area targeted for cellulite reduction using a predetermined combination of massage techniques. Cellulite reduction results from the combined laser or light and massage applications. Other tissue treatments are possible with variation of the laser or light and massage applications. The details of an exemplary system that may be used in the methods of the invention can be found in, for example, U.S. Pat. No. 6,673,096B2, the entire contents of which are incorporated by reference herein.

Beneficial effects may be achieved using wavelengths in the 600-950 nm wavelength range, with the exception of 700-770 nm wavelengths range which is generally considered ineffective. Hence the choice of substantially monochromatic wavelength is an important consideration when considering treatment of superficial or deeply located tissues—shorter wavelengths for more superficial applications and longer wavelengths for deeper penetration.

It should be understood that numbers intermediate to any wavelength ranges recited herein, e.g., 640, 641, 643, 643, 644, 645, 646, 647, 648, 649, 650, 651, 652, 653, 654, 655, 666, 667, 668, 669, and 670 nm, are also intended to be part of this invention. Ranges of values using a combination of any of the above recited values as upper and/or lower limits are also intended to be included in the scope of the invention

In one embodiment, the duration, power, and wavelengths of the low intensity light depends on various factors, such as calf circumference, thigh circumference, abdominal circumference, hip circumference, patient weight, and patient height. For example, a patient having a thigh circumference of 38 cm, calf circumference of 31 cm, abdominal circumference of 76 cm, hip circumference of 82 cm, weight of 62 kg, ideal body weight of 50 kg according to Standard Metropolitan Life Insurance Company Tables, and height of 5 feet 3 inches, would be exposed to the radiation for 6.4 minutes per area. The time duration for exposure of each area on the skin surface 14 is calculated based on the following expression: (Hip Circumference in cm)/(Abdominal Circumference in cm)×(Weight in kgs)/(Ideal Body Weight in kgs)×(Calf Circumference in cm+Thigh Circumference in cm)×Reduction Factor=Time in Minutes. Ideal Body Weight (“IBW”) is as defined by Standard Metropolitan Life Insurance Company tables for IBW calculated from the patient's height.

An exemplary massaging machine used in the methods of the invention is a machine made by Spa-de-Soleil. Alternatively, the light emitting component and the massage component can be combined into a single hand operated device, such as described in U.S. Pat. No. 6,673,096B2, the entire contents of which are incorporated by reference herein.

In short, an example system suitable for implementing the method of the present invention applies laser radiation to an area targeted for, e.g., cellulite and superficial fat reduction, in a predetermined wavelength, or combination of wavelengths, intervals, periods, and patterns. The application of infrared radiation can optionally follow. Example wavelengths include about 650 nm and about 915 nm. The tissue treatment system also massages the area that was illuminated by the laser radiation using a predetermined combination of massage techniques, including repeating lateral movements back and forth across the treatment area. Cellulite reduction results from the combined laser and massage applications. Other tissue treatments are possible with variation of the laser and massage applications.

In one embodiment, a method of the present invention is used for reducing cellulite and superficial fat in a subject in need thereof. The method includes treating a subject with a first wavelength of low intensity light (for example, about 650 nm) for a duration of time that is sufficient for increasing the permeability of cell membranes of adipocytes. The period of time is specific to the treatment, and can be calculated as described above. At least a second wavelength (for example, about 915 nm) or a plurality of distinct wavelengths of low intensity light are applied for a duration of time that is sufficient for causing liquefaction of fat in the adipocytes, thereby resulting in cellulite reduction. The example calculation above demonstrates one possible time period. In one embodiment, the subject is exposed to the first wavelength, the second wavelength, and any subsequent wavelengths sequentially, e.g., the first wavelength is used first followed by the second wavelength, followed by a third, etc., or alternating, e.g., the second wavelength is used first, followed by the first wavelength, switching back and forth in an alternating manner. In another embodiment, the first wavelength, the second wavelength, and any additional wavelengths are used simultaneously to achieve the desirable result, e.g., cellulite and fat reduction.

In accordance with one embodiment of the present invention, the application of the laser radiation results in a moderate increase in tissue temperature. For example, an acceptable range for the moderate increase is up to about 102° F. to about 106° F. It is contemplated that any suitable means of increasing tissue temperature to the desired range may be used in the methods of the invention.

The low intensity light used in the methods of the invention in accordance with some embodiments may be continuous, and in accordance with other embodiments may be pulsed, so long as it achieves the desired result. The radiation-emitting device can also emit radiation in the visible (e.g., light) and/or infrared wavelengths, for example at about 650 nm and about 900+/−20 nm, which includes about 915 nm. In one example embodiment, the radiation-emitting device emits radiation in a concentric combination of infrared and visible laser light. Alternatively, the wavelengths can be emitted by LEDs or equivalent light emitting devices in the visible and/or infrared light ranges. In one embodiment, the method of the invention includes a dual-band light therapy (including, but not limited to, 650 nm red light from laser diodes and LED with near infrared 900+20 nm, which includes about 915 nm, from LED) with a mechanical massager and suction, including, but not limited to, vacuum.

In accordance with the example embodiment of the present invention, the method further includes the application of massage to the tissue being treated. In accordance with one embodiment, an automated vacuum mechanical massaging mechanism is utilized. The step of massaging the exposed skin can be executed, for example, by massaging from points distal from a heart of the patient to points proximal to the heart of the patient to encourage fluid flow toward the heart. Further, the step of massaging the exposed skin can include massaging in predetermined patterns of motion.

The present invention provides a synergistic effect, wherein the combination of the low intensity light therapy and the massage system acts to reduce cellulite and superficial fat. The massage system used in the methods of the present invention may include a vacuum device, a mechanical device, or both.

In accordance with various aspects of the present invention, the method implemented herein can result in improving local blood and lymphatic circulation; improving metabolic activity of cells; increasing collagen production; reducing inflammation; improving cell survival; and/or increasing tissue oxygenation. Accordingly, in a method according to the present invention, one or more of these therapeutic effects are achieved by treating a subject with a first wavelength of low intensity light and/or a second wavelength of low intensity light, for a duration of time sufficient to achieve one or more of the aforementioned therapeutic effects.

The reduction in cellulite can be a reduction in an amount or the appearance of cellulite in the subject being treated according to the methods of the invention. Further the reduction can be partial decrease in the amount or appearance of cellulite or a complete disappearance in the cellulite, following treatment using the methods of the invention. Also, the reduction may be achieved in a single treatment or multiple treatments and the number of treatments desired for a particular subject can be readily determined by one of ordinary skill in the art. Exemplary methods for determining reduction in cellulite are provided herewith in the Examples.

In one embodiment, a reduction in cellulite may be characterized using the Cellulite Classification Tool (CCT) described below in Example 2. The CCT is based on a cellulite classification score determined by grading three different parameters of cellulite in a subject: overall skin appearance, projections/bumps, and dimpling/depressions. Based one subscores ranging from 0 to 3 for each of these subcategories, a total score is determined. The total score ranges from 0 (no cellulite) to severe (scores 7-9). Thus, in one embodiment, the invention provides a method for improving the CCT score of a subject using the methods described herein. In one embodiment, the improvement is a decrease of 1, 2, 3, 4, 5, 6, 7, 8, or 9 according to the CCT scale. For example, in one embodiment, a subject has a decrease of 1 according to the CCT scale, where the pre-treatment score is 7 and the post-treatment score is 6. In another embodiment, the improvement is no increase (0) according to the CCT scale. For example, a subject has a pre-treatment score of 3 and the post-treatment score of 3. An improvement of 0 would indicate that cellulite had been prevented, as the condition had not worsened. Alternatively, the CCT scale may be used to describe a reduction in cellulite using the methods of the invention based on the general classifications of “No cellulite” (score 0), “Mild cellulite” (score 1, 2, or 3), “Moderate cellulite” (score 4, 5, or 6), or “Severe cellulite” (score 7, 8, or 9). As such, the methods of the invention may be used to improve the CCT score of a subject from Severe to Moderate, Severe to Mild, or Severe to No Cellulite. Alternatively, the methods of the invention may be used to improve the CCT score of a subject from Moderate to Mild, Moderate to No Cellulite, or Mild to No Cellulite. In another alternative, the methods of the invention may be used to maintain the CCT score of a subject, e.g., maintain a “No cellulite” score or a Mild score. In some embodiments it may also be beneficial to the subject to use the methods of the invention to maintain a Moderate score according to the CCT scale.

In one embodiment, the invention also includes a method of determining reductions in cellulite formation comprising using the Cellulite Classification Tool (CCT) described herein. In one embodiment, the CCT may be applied to the treatment methods of the invention to determine a reduction in cellulite. Alternatively the CCT may be applied to other methods known in the art for improving cellulite. Also encompassed within the invention are kits or articles of manufacture that comprise a printed material, e.g., package insert, label, or pamphlet, which describes the scaling system of the CCT.

In addition to reducing cellulite, the methods of the invention employing the use of the first and/or second wavelengths may also be used for the treatment of other medical conditions. Exemplary conditions include, but are not limited to, for example, wounds, arthritis, neuropathies, inflammation, edema, and intractable pain. Without wishing to be bound by theory, it is contemplated that various therapeutic effects of low intensity light therapy result from modulation of one or more molecular and cellular parameters selected from: mitochondrial function, transcription factors, cell survival, blood flow, inflammation, calcium dynamics and various systemic effects. Accordingly, in one embodiment, a desirable result may be achieved by modulation of one or more of the cellular and/or molecular parameters using the methods of the invention, for example, by one or more of: improving mitochondrial function, activating transcription factors, improving cell survival, improving blood flow, reducing inflammation, modulating calcium dynamics, and/or altering endorphin/serotonin nociception.

Additional energy sources may also be used in the methods of the present invention including, but not limited to, for example, radio frequency (RF), electrical stimulation (ES), and ultrasound (focused and/or collimated).

As will be appreciated by the skilled artisan, the frequency of performing the method of the invention on a subject will vary depending upon the desired results and/or the amount of cellulite present in the target area. Exemplary treatment schedules are provided herewith in Example 1.

Numerous modifications and alternative embodiments of the invention will be apparent to those skilled in the art in view of the foregoing description. Accordingly, this description is to be construed as illustrative only and is for the purpose of teaching those skilled in the art the best mode for carrying out the invention. Details of the structure may vary substantially without departing from the spirit of the invention, and exclusive use of all modifications that come within the scope of the appended claims is reserved. It is intended that the invention be limited only to the extent required by the appended claims and the applicable rules of law.

The specification is most thoroughly understood in light of the teachings of the references cited within the specification which are hereby incorporated by reference. The embodiments within the specification provide an illustration of embodiments in this application and should not be construed to limit its scope. The skilled artisan readily recognizes that many other embodiments are encompassed by this disclosure. All publications and patents cited and sequences identified by accession or database reference numbers in this disclosure are incorporated by reference in their entirety. To the extent that the material incorporated by reference contradicts or is inconsistent with the present specification, the present specification will supercede any such material. The citation of any references herein is not an admission that such references are prior art to the present specification.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. Unless otherwise indicated, the term “at least” preceding a series of elements is to be understood to refer to every element in the series.

Those skilled in the art will recognize, or be able to ascertain using no more than routine experimentation, many equivalents to the specific embodiments of the invention described herein. Such equivalents are intended to be encompassed by the following claims. This invention is further illustrated by the following example which should not be construed as limiting. The contents of all references, patents and published patent applications cited throughout this application are incorporated herein by reference.

### EXAMPLES

### Example 1

### Study Evaluating Method for Reducing Cellulite

The following example describes a study which examined the benefits, including the reduction of cellulite, using the methods of the invention. A combination method using dual-band light at 650 nm & 915 nm wavelengths was combined with massage and suction. The 915 nm wavelength was preferentially absorption in lipids leading to adipocytes' moderate temperature elevation and fat liquefaction, whereas the 650-nm light altered permeability of the adipocytes' membrane. Vacuum massage assisted in evacuation of liquefied fat from intracellular space.

10 female volunteers with mild-moderate cellulite received 8 twice-a-week (i.e., two treatments per week for four weeks) treatments using the dual band light treatment in combination with vacuum massage. Bilateral body sites were treated with either 11 W of combined optical power (1 W @ 650 nm & 10 W @ 915 nm) on one side or 0.6W (0.02@ 650 nm & 0.58 @ 915 nm) on other.

Reduction in cellulite was evaluated using the following means: anthropometric measurements (e.g., thigh circumference), bioelectrical impedance analysis (e.g., BMI, Fat vs. Muscle ratio), digital clinical photography, high frequency ultra-sound imaging, blinded evaluators, and subjects self evaluation questionnaire.

Digital clinical photography was performed using blinded evaluators prior to the treatment, as well as at regular intervals following the treatment, including 3 days post-treatment, 1 month post-treatment, 3 months post-treatment.

Results from patients who completed treatment and follow up analysis exhibited a number of unexpected improvements. Improvements in the overall appearance of the subjects skin was observed following treatment. Visual improvements included a reduction in the bumpiness and improved elasticity, were observed following treatments at the various time points.

On the low power side 50% of subjects reported improvements in cellulite:
10% at the end of treatment
25% at 1 month follow up
40% at 3 months follow up
   Subjects also reported the high-power side to be smoother looking and firmer after 5-6 treatments. 80% of subjects demonstrating improvement in cellulite as evaluated by “blinded” observers. 40% showed a reduction in cellulite at the end of treatment, 50% showed a reduction in cellulite at 1 month follow up, and 60% showed a reduction in cellulite at the 3 month follow up.

The self-evaluation results showed that subjects were greatly satisfied with the treatment, and were satisfied with the improvement in cellulite reduction.

The conclusion of the study was that the dual band light treatment of 650 and 915 nm in combination with massage were effective at treating cellulite and improving the overall appearance of the treated area.

### Example 2

### Cellulite Classification Tool (CCT)

The following example describes a tool for classifying improvements in cellulite. Certain steps are performed to obtain data to be used in the following Cellulite Classification Tool (CCT):
Step 1: Determine 8×10″ target area and assess this area only
Step 2: Visually inspect skin and assess for irregularities
Step 3: Perform pinch test by holding skin between the thumb and other fingers, gently lift skin while applying slight pressure and assess for skin irregularities
Step 4: Have subject lie down and assess skin for irregularities
Step 5: Read each category of the CCT and determine which score in each category best describes the subject
Step 6: Total all scores and determine cellulite class

The different categories and the scores for each are provided in Table 1 below:

TABLE 1
CCT Categories and Scoring System

| | | | | |
|---|---|---|---|---|
| Score | 0 | 1 | 2 | 3 |

Overall Skin Appearance

| | | | | |
|---|---|---|---|---|
| General look | Smooth, firm | Mostly smooth | Skin with | Majority of skin with |
| of skin | skin. Pinch test | skin, minor | irregular texture, | very irregular texture. |
| | reveals no | areas of | bumps and | Prevalent bumps and |
| | mattress-like | irregular texture. | round or linear | round and/or linear |
| | appearance. | Pinch test | depressions. | depressions. Cellulite |
| | No surface | reveals mattress | Some loose skin | present when standing |
| | irregularities | like appearance. | noted. Cellulite | and lying down. |
| | noted when | No dimpling | visible when | |
| | standing or | when patient is | standing but not | |
| | lying down. | standing or | lying down. | |
| | | lying down. | | |

Projections/Bumps

| | | | | |
|---|---|---|---|---|
| Protrusion noted | No bumps - | Mild | Pronounced | Severe bumpiness- |
| at the surface of | smooth, firm | projections- | bumpiness- | large, highly elevated |
| the skin | skin. | small, low | moderately | protrusions. >10 in |
| | | profile. <5 | elevated | assessment area. |
| | | noted in | protrusions. 5- | |
| | | assessment area. | 10 in assessment | |
| | | | area. | |

Dimpling/Depressions

| | | | | |
|---|---|---|---|---|
| Round or linear | No dimples - | Slight dimpling- | Pronounced | Severe dimpling - large |
| depression noted | smooth, firm | small, shallow | dimpling- | deep depressions >10 in |
| on the surface | skin. | depressions <5 | medium, deep | assessment area |
| of the skin | | noted in | depressions 5-10 | |
| | | assessment area | in assessment | |
| | | | area | |

Based on the scoring system described in Table 1, a total score is determined for the subject. The cellulite classification is then determined according to the system described in Table 2:

TABLE 2
CCT Cellulite Classification
Total Score = Cellulite Classification

| No Cellulite | Mild | Moderate | Severe |
|---|---|---|---|
| 0 | 1-3 | 4-6 | 7-9 |
| | 1 - Mild A | 4 - Moderate A | 7 - Severe A |
| | 2 - Mild B | 5 - Moderate B | 8 - Severe B |
| | 3 - Mild C | 6 - Moderate C | 9 - Severe C |

The cellulite score of a subject is determined prior to treatment and then at a time point(s) following treatment to determine whether an improvement is observed

1. A method for reducing cellulite in a targeted area of a subject in need thereof comprising:
treating the targeted area of the subject with a first wavelength of low intensity light for a duration of time that is sufficient for increasing the permeability of cell membranes of adipocytes; and at least a second wavelength of low intensity light for a duration of time that is sufficient for causing liquefaction of fat in the adipocytes, wherein the treatment with the first and at least the second wavelengths of low intensity light results in cellulite reduction in the targeted area of the subject.

2. The method of claim 1, wherein the first and at least the second wavelengths of low intensity light are used sequentially or alternating.

3. The method of claim 1, wherein the first and at least the second wavelengths of low intensity light are used simultaneously.

4. A method for reducing cellulite in a targeted area in a subject in need thereof comprising:
(a) treating the targeted area with a first wavelength of low intensity light for a duration of time that is sufficient for increasing the permeability of cell membranes of adipocytes; and at least a second wavelength of low intensity light for a duration of time that is sufficient for causing liquefaction of fat in the adipocytes; and
(b) causing a moderate increase in tissue temperature in the targeted area, thereby resulting in cellulite reduction.

5. A method of reducing cellulite in a subject in need thereof comprising:
(a) treating the subject with a first wavelength of low intensity light for a duration of time that is sufficient for increasing the permeability of cell membranes of adipocytes; and at least a second wavelength of low intensity light for a duration of time that is sufficient for causing liquefaction of fat in the adipocytes; and
(b) using a massage system on the subject,
wherein the combination of (a) and (b) results in cellulite reduction in the subject.

6. A method of achieving one or more therapeutic effects selected from the group consisting of: improving local blood and lymphatic circulation; improving metabolic activity of cells; increasing collagen production; reducing inflammation; improving cell survival; and increasing tissue oxygenation, in a subject, the method comprising: treating the subject with a first wavelength of low intensity light and/or at least a second wavelength of low intensity light, for a duration of time sufficient to achieve one or more of the therapeutic effects.

7. The method of claim 1, wherein the duration of time used in the methods of the invention is a predetermined duration of time.

8. The method of claim 1, further comprising massaging the targeted area of the subject.

9. The method of claim 1, wherein the first and at least the second low intensity light wavelengths used in the methods of the invention are predetermined wavelengths.

10. The method of claim 1, wherein the first wavelength of low intensity light is about 650 nm.

11. The method of claim 1, wherein at least the second wavelength of low intensity light is about 915 nm.

12. The method of claim 1, wherein the first and/or at least the second wavelength of low intensity light is a continuous light of fixed intensity.

13. The method of claim 1, wherein the first and/or at least the second wavelength of low intensity light is in the form of a modulated continuous wave light.

14. The method of claim 1, wherein the first and/or at least the second wavelength of low intensity light is a pulsed light.

15. The method of claim 5, wherein the massage system comprises a vacuum massage device.

16. The method of claim 5, wherein the massage system comprises a mechanical massage device.

17. The method of claim 5, wherein the effect is synergistic.

18. The method of claim 8, wherein the massaging vacuum massaging.

19. A method for reducing or preventing cellulite or superficial fat in a targeted area of a subject, said method comprising
a) increasing the permeability of a cell membrane of an adipocyte of the targeted area;
b) increasing the temperature of the adipocyte such that lipid liquefaction occurs; and
c) evacuating liquefied fat from the adipocyte,
such that cellulite or superficial fat in the targeted area of the subject is reduced or prevented.

20. A method of achieving one or more therapeutic effects selected from improving skin elasticity, reducing a circumference, and increasing collagen production in a treated area of a subject, said method comprising
a) increasing the permeability of a cell membrane of an adipocyte of the targeted area;
b) increasing the temperature of the adipocyte such that lipid liquefaction occurs; and
c) evacuating liquefied fat from the adipocyte.

21. The method of claim 19, wherein step (a) comprises treating the targeted art of the subject with a wavelength of low intensity light.

22. The method of claim 21, wherein the wavelength of low intensity light ranges from about 600 to about 699 nm.

23. The method of claim 22, wherein the wavelength of low intensity light is about 650 nm.

24. The method of claim 19, wherein step (b) comprises treating the targeted art of the subject with a wavelength of low intensity light

25. The method of claim 24, wherein the wavelength of low intensity light ranges from about 900 to 950 nm.

26. The method of claim 25, wherein the wavelength of low intensity light is about 915 nm.

27. The method of claim 19, wherein step (c) comprises using a massage system on the treated area of the subject.

28. The method of claim 27, wherein the massage system comprises a vacuum massage system.

29. The method of claim 19, wherein the reduction or prevention of cellulite or superficial fat improves over the course of at least 1 month or over the course of at least 2 months.

30. (canceled)

31. The method of claim 19, wherein the reduction or prevention of cellulite or superficial fat improves over the course of at least 3 months.

32. The method of claim 20, wherein the therapeutic effect improves over the course of a time period selected from the group consisting of at least 1 month, at least 2 months, and at least 3 months.

33-34. (canceled)

35. The method of claim 4, wherein the duration of time used in the methods of the invention is a predetermined duration of time.

36. The method of claim 4, further comprising massaging the targeted area of the subject.

37. The method of claim 4, wherein the first and at least the second low intensity light wavelengths used in the methods of the invention are predetermined wavelengths.

38. The method of claim 4, wherein the first wavelength of low intensity light is about 650 nm.

39. The method of claim 4, wherein at least the second wavelength of low intensity light is about 915 nm.

40. The method of claim 4, wherein the first and/or at least the second wavelength of low intensity light is a continuous light of fixed intensity.

41. The method of claim 4, wherein the first and/or at least the second wavelength of low intensity light is in the form of a modulated continuous wave light.

42. The method of claim 4, wherein the first and/or at least the second wavelength of low intensity light is a pulsed light.

43. The method of claim 5, wherein the duration of time used in the methods of the invention is a predetermined duration of time.

44. The method of claim 5, wherein the first and at least the second low intensity light wavelengths used in the methods of the invention are predetermined wavelengths.

45. The method of claim 5, wherein the first wavelength of low intensity light is about 650 nm.

46. The method of claim 5, wherein at least the second wavelength of low intensity light is about 915 nm.

47. The method of claim 5, wherein the first and/or at least the second wavelength of low intensity light is a continuous light of fixed intensity.

48. The method of claim 5, wherein the first and/or at least the second wavelength of low intensity light is in the form of a modulated continuous wave light.

49. The method of claim 5, wherein the first and/or at least the second wavelength of low intensity light is a pulsed light.

50. The method of claim 6, wherein the duration of time used in the methods of the invention is a predetermined duration of time.

51. The method of claim 6, wherein the first and at least the second low intensity light wavelengths used in the methods of the invention are predetermined wavelengths.

52. The method of claim 6, wherein the first wavelength of low intensity light is about 650 nm.

53. The method of claim 6, wherein at least the second wavelength of low intensity light is about 915 nm.

54. The method of claim 6, wherein the first and/or at least the second wavelength of low intensity light is a continuous light of fixed intensity.

55. The method of claim 6, wherein the first and/or at least the second wavelength of low intensity light is in the form of a modulated continuous wave light.

56. The method of claim 6, wherein the first and/or at least the second wavelength of low intensity light is a pulsed light.

57. The method of claim 20, wherein step (a) comprises treating the targeted art of the subject with a wavelength of low intensity light.

58. The method of claim 57, wherein the wavelength of low intensity light ranges from about 600 to about 699 nm.

59. The method of claim 58, wherein the wavelength of low intensity light is about 650 nm.

60. The method of claim 20, wherein step (b) comprises treating the targeted art of the subject with a wavelength of low intensity light

61. The method of claim 50, wherein the wavelength of low intensity light ranges from about 900 to 950 nm.

62. The method of claim 61, wherein the wavelength of low intensity light is about 915 nm.

63. The method of claim 20, wherein step (c) comprises using a massage system on the treated area of the subject.

64. The method of claim 63, wherein the massage system comprises a vacuum massage system.
